(19) United States
(12) Patent Application Publication
(10) Pub. No.: US 2025/0288288 A1
(43) Pub. Date: Sep. 18, 2025

(54) RETRACTOR SYSTEM FOR ABDOMINAL-WALL REPAIR

(71) Applicant: The Cleveland Clinic Foundation, Cleveland, OH (US)

(72) Inventors: Michael Rosen, Solon, OH (US); Nir Messer, Beachwood, OH (US); Walter Zimmer, Chagrin Falls, OH (US)

(21) Appl. No.: 19/077,527

(22) Filed: Mar. 12, 2025

Related U.S. Application Data
(60) Provisional application No. 63/564,106, filed on Mar. 12, 2024.

(51) Int. Cl. A61B 17/02; A61B 17/00

(52) U.S. Cl. CPC A61B 17/0281; A61B 2017/00407

(57) ABSTRACT

An abdominal-wall retractor includes a mounting armature affixed stationary relative to or near an operating table. The retractor includes an upward tensioning rail affixed to the mounting armature. An upward tension linkage can engage the upward tension rail and provide tension between the upward tensioning rail and an upward surgical tool affixed to an incision site of the patient. The mounting armature includes one or more arms to elevate and position the upward tensioning rail in a fixed position above a surgical site in order to retract an abdominal wall upward at an angle from the horizontal via the supplied tension in the upward tension linkage during a surgical procedure. A lateral retraction rail can be positioned on the mounting armature below the upward tension rail. A lateral tension linkage with a lateral surgical tool can engage the lateral retraction rail to provide lateral retraction to the abdominal wall.

### CROSS-REFERENCE TO RELATED APPLICATIONS

This application claims the benefit of U.S. provisional patent application No. 63/564,106 filed Mar. 12, 2024, which is incorporated herein by reference.

### BACKGROUND

Abdominal wall reconstruction (“AWR”) is a common surgical procedure performed in both emergency and elective situations. Approximately 300,000 repairs are performed in the United States annually. A typical elective procedure aims to repair an abdominal-wall hernia, whereas an emergency one can be to treat a patient who has suffered an acute event—typically having a piece of intestine incarcerated within the defect with the potential for strangulation and perforation necessitating immediate intervention. In both situations, suitable retraction of the abdominal wall is required in order to provide surgical access to the underlying fascial layers to perform the necessary steps to repair the hernia defect. These layers include the linea alba, or the midline fascial insertion of the underlying muscles and the posterior rectus sheath, the covering of the rectus muscle. Additionally, lateral exposure of the oblique muscles can be required for larger hernia repairs. Current retractors do not provide exposure to these abdominal wall structures to permit safe and effective retraction to facilitate a hernia repair. In fact, all available retraction systems are designed to specifically retract the abdominal wall out of the field to provide adequate exposure for the visceral organs to perform gastrointestinal surgery. As an example, the Bookwalter retractor system (shown in FIGS. 1A and 1B) is typically used to retract the abdominal wall to provide access for operating on internal organs. The Bookwalter retractor is not suitable to provide retraction for AWR procedures because it does not provide surgical access to the underlying fascia. Rather, the circular rail of a Bookwalter retractor is arranged essentially tangent to the level of the surgical incision, and the clamps and other instruments affixed to it retract the abdominal wall generally within the plane of that rail, i.e. radially outward from its center, to provide the largest practical opening through the abdominal wall to access the surgical field below.

But such purely radial-outward retraction of the abdominal wall is not useful in AWR procedures because it does not provide access to the underlying fascial tissue of the abdominal wall. To perform an AWR procedure the abdominal wall must initially be retracted upward, lifting the dissected flap of abdominal-wall tissue so that the surgeon can operate directly on the underlying fascia or posterior rectus sheath. Currently, to supply the required upward retraction to allow surgical access, surgical assistants manually lift the abdominal wall from the incision site and hold it upward or at an upward angle. Initially, this upward retraction is supplied by surgical assistants placing clamps (Lahey clamps) on the fascial edges and pulling upward. For eventual lateral exposure, the assistants will switch to a Richardson retractor to provide upward and outward retraction for the lateral extremes of the procedure.

Assistants must sustain this manual retraction for the entire AWR procedure. And the magnitude of retraction typically must be increased as the surgery proceeds. Increased retraction is necessary to provide access to deeper regions of the abdominal fascia, which often will be dissected from the anterior surgical opening all the way up to just adjacent to the spine posteriorly. The continuous application of such manual, increasing-force retraction, produces significant physical stress and fatigue in the surgical assistants. These stresses, incurred for several hours at a time per procedure, at a rate of up to six procedures per week, can yield orthopedic injury to the fingers, elbows, shoulders, and neck of the assistants over time. Further, as the assistants become fatigued, the applied traction may become diminished, thus obscuring the surgical field for the operator and decreasing the efficiency of the procedure.

Additionally, during AWR procedures, as fascia is dissected from the rectus and transversus abdominus muscles of the abdomen, it may be desirable to supply counter-tension to these dissected parts. For instance, the surgeon or assistant may need to apply tension in a direction that tends to stretch the dissected posterior rectus fascia taut and away from the retracted abdominal wall, or toward the surgeon. Maintaining tautness (i.e., planar tension) in the dissected fascia can be important as the surgeon continues to operate and further dissect it from the rectus and transversus abdominus muscles in hernia surgery. Currently, this counter tension is provided by the surgeon's non-operative hand. Applying a consistent and reliable counter-tension may be difficult for the surgeon based on strength and dexterity of the surgeon's hand, the attention to the his/her operative hand, and space around the surgeon during the procedure.

### BRIEF SUMMARY

A retractor system is disclosed that provides persistent, effective abdominal-wall retraction for AWR procedures that does not require sustained manual pulling of the abdominal wall, in a manner that exposes the underlying fascia as required for completing an operation.

In accordance with one aspect, an abdominal-wall retractor may include a mounting armature and an upward tensioning rail affixed to the mounting armature. One or more upward tension linkages can be configured to provide tension between the upward tensioning rail and one or more respective upward retraction tools. The mounting armature can be configured to hold the upward tensioning rail in a fixed position above a surgical site in order to retract an abdominal wall upward, when grasped by the upward retraction tool(s), at an angle of at least 20° from horizontal via the provided tension in the one or more upward tension linkages during a surgical procedure.

In accordance with another aspect, an abdominal-wall retractor system may include a mounting armature, an upward tensioning rail affixed to the mounting armature, and a lateral tensioning rail affixed to the mounting armature at a location lower than the upward tensioning rail. The system may include an upward tension linkage configured to provide upward tension between the upward tensioning rail and an upward retraction tool along an upward retraction vector. The system may also include a lateral tension linkage configured to provide lateral tension between the lateral tensioning rail and a lateral retraction tool along a lateral retraction vector. The upward retraction vector can be at a greater angle than the lateral retraction vector relative to horizontal.

In accordance with a further aspect, a method for abdominal-wall repair, comprises applying an upward tension to an abdominal wall flap via an upward tension linkage extending between an upward tension rail and the abdominal wall flap along an upward retraction vector at an angle of at least 20° relative to horizontal, in order to expose underlying fascia of the abdominal wall flap.

### BRIEF DESCRIPTION OF THE DRAWINGS

FIG. 1A illustrates a conventional Bookwalter retractor system.

FIG. 1B illustrates the conventional Bookwalter retractor system in use.

FIG. 2 illustrates a first embodiment of an abdominal-wall retractor system as herein disclosed.

FIG. 3 illustrates a second embodiment of an abdominal-wall retractor system as herein disclosed, this time in use during an AWR procedure.

FIG. 3a shows conventional “cricket” Kocher clamps, which may be used as described herein.

FIG. 3b shows example clasps welded to the distal ends of ball chains for affixation to respective clamps as herein described.

FIGS. 4a-b show the abdominal-wall retraction system supplying both upward and lateral tension in-use.

FIG. 5 shows a rear view of an embodiment of the abdominal-wall retraction system wherein an example mounting armature is clearly shown.

FIG. 6 shows a third embodiment of the abdominal-wall retraction system.

FIG. 7 shows a first embodiment of a counter-tension clamping assembly.

FIG. 8 shows a second embodiment of a counter-tension clamping assembly.

FIG. 9 shows the first embodiment of a counter-tension clamping assembly being used to provide counter-tension to dissected fascia in an AWR procedure.

### DESCRIPTION OF EXAMPLE EMBODIMENTS

A retractor system, as disclosed, provides persistent, effective abdominal-wall retraction for AWR procedures that does not require sustained manual pulling of the abdominal wall, in a manner that exposes the underlying fascia as required for completing an operation. The retractor system may provide the upward and lateral traction typically required for AWR procedures.

During AWR repair it is important to retract the dissected abdominal wall upward in a manner that provides access to the underlying abdominal fascia, which in AWR procedures is, or constitutes a significant part of, the surgical field. By “upward,” it is not necessary that retraction must be vertically upward along a direction perpendicular to the horizontal. Rather, “upward” means that retraction of the abdominal wall from the incision site is along a vector that includes a substantial vertical component, for example at an angle of at least 20°, preferably at least 30°, 40° or 45°, or even up to 60° from the horizontal. For example, such an upward vector can be in the range of 20° to 60°, 30° to 60°, or 40° to 60° from the horizontal. Larger-angle upward vectors lower than 90° from the horizontal are also possible. As will be appreciated, the angle of the upward vector depends on the placement of the retractor system relative to the incision site, as further described below. Such upward retraction vectors ensure that the abdominal wall is retracted upward to a substantial degree, and not merely or predominantly laterally outward (along the horizontal) from the incision site as is the case using a conventional Bookwalter retractor. Such upward retraction will provide sufficient surgical access to the abdominal fascia and associated tissues for dissection.

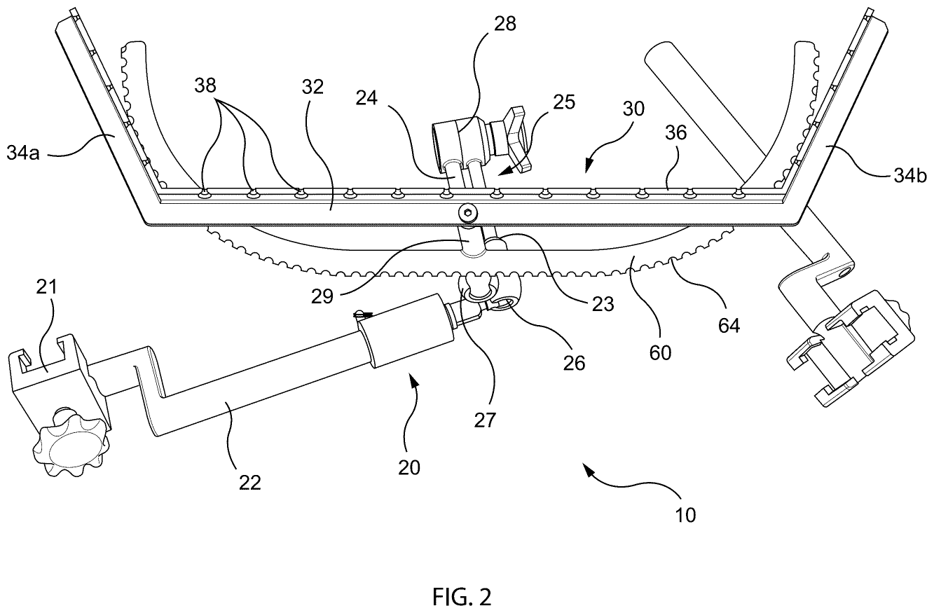

Referring first to FIG. 2, an embodiment of the abdominal-wall retractor system 10 is shown, which includes a mounting armature 20 and an upward tensioning rail 30. The retractor system 10 may also include a lateral tensioning rail 60. The mounting armature 20 is configured to fixedly and adjustably position the upward tensioning rail 30 and lateral tensioning rail 60 at a desired location and in a desired orientation relative to the surgical field. For instance, the mounting armature 20 may fixedly position the upward tensioning rail 30 and/or the lateral tensioning rail 60 generally above the surgical field (or the abdominal-wall incision) and opposite to the operator during an AWR procedure. The mounting armature 20 is configured to be affixed to a complimentary and stationary anchor point relative to the surgical field, which can be on a surgical bed or on a vertical stand positioned near the operating table.

In the embodiment shown in FIG. 2, the mounting armature 20 includes a clamp 21 configured to affix to a post (e.g., a bedpost), which itself can be affixed or at least stationary relative to the operating table. A horizontal armature 22 extends from the clamp and is coupled to the first arm 23 of a scissor linkage 25 via a first universal ball joint 26. The first arm 23 is coupled at its opposite end to the second arm 24 of the scissor linkage 25 via an articulated scissor joint 28. A vertical post 29 is coupled to and extends from the second arm 24 via a second universal ball joint 27 at the end of the second arm 24 opposite to where it is articulated to the first arm 23. The upward tensioning rail 30 is affixed to the vertical post 29 as shown. The upward tensioning rail 30 may be secured to the vertical post 29 with a fastener, a rivet, pin, or some other suitable mechanical fastener. The upward tensioning rail 30 may also be welded to the vertical post 29. Alternatively, the upward tensioning rail may be removably and selectively attached to the vertical post 29.

With the example structure of the mounting armature 20 as described above, it will be appreciated that the operator may mount the armature 20 to a fixed vertical post via the clamp 21, and then articulate the linkages of the armature 20 in order to position the upward tensioning rail 30 above the surgical field (or abdominal-wall incision site) at a desired location and in a desired orientation relative to the surgical field. For instance, in order to provide upward retraction of the abdominal wall, as will be described below, the upward tensioning rail 30 can be positioned above the incision site and opposite to where the operator will work. The upward tensioning rail 30 is generally placed opposite the operator to prevent interference with the operator during the procedure. In the illustrated embodiment, articulation of the described linkages of the armature 20 is achieved by adjusting the respective ball joints 26, 27 and the articulated scissor joint 28, all of which can be loosened for adjustment and then locked. For instance, the ball joints 26, 27 and articulated scissor joint may be locked via a set screw or compression tightening once the desired degree of adjustment for each has been achieved. It will be appreciated that the ball joints provide a wide range of movement that can allow for fine or precise positioning of the upward tensioning rail 30 relative to the incision site. The direction from which the armature 20 generally extends from the mounting post also can be adjusted by rotating thereabout via the clamp 21, and then tightening the clamp 21 once the desired direction of extension has been achieved.

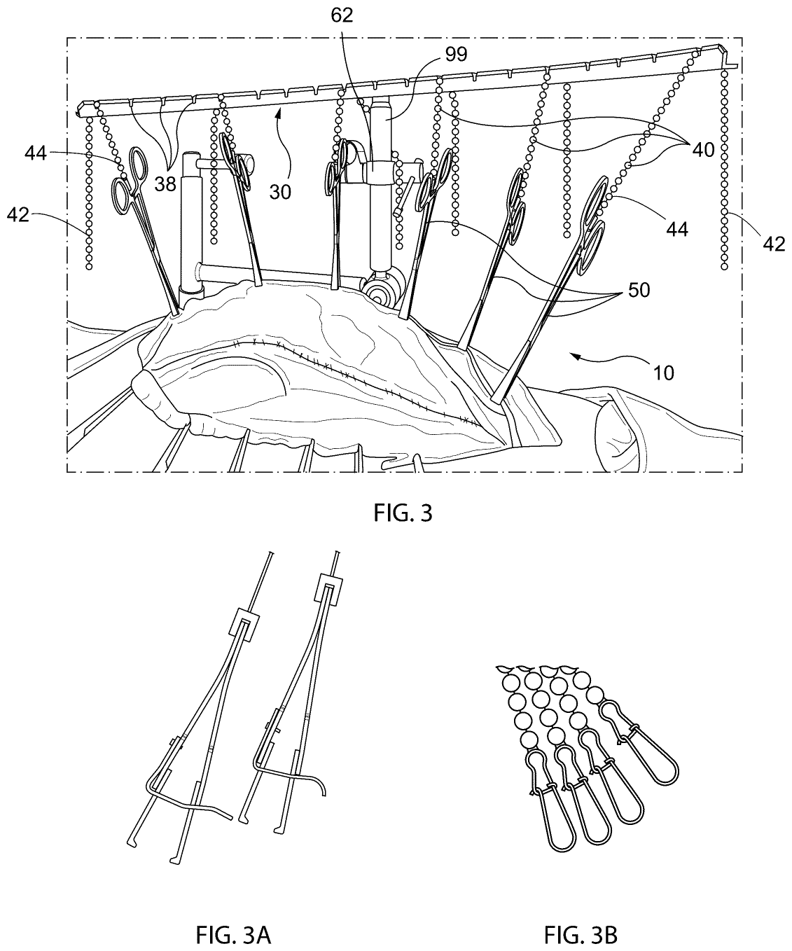

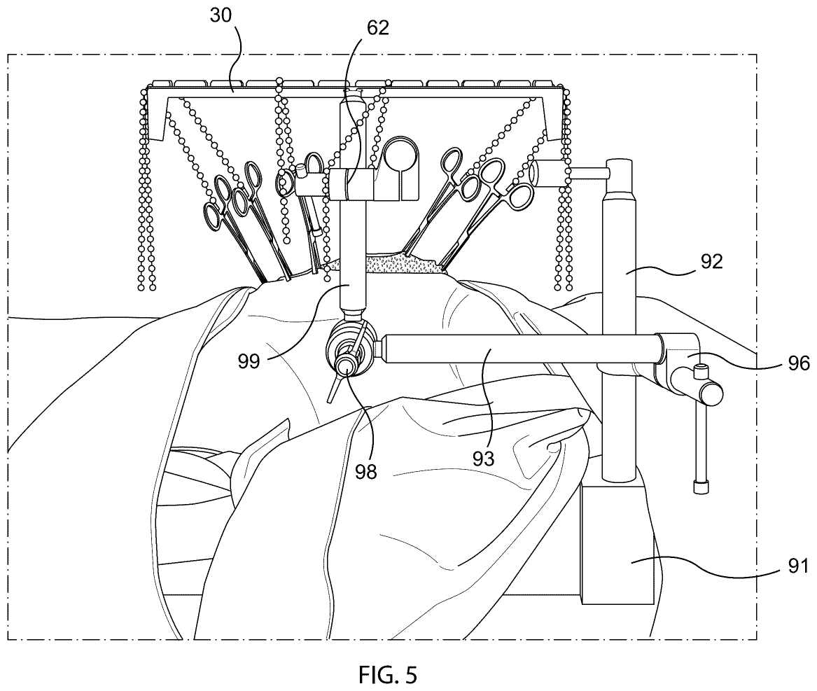

FIGS. 3-5 illustrate another embodiment of the abdominal-wall retractor system 10 having an alternative mounting armature 90, in-use during an AWR procedure. This embodiment differs from the one shown in FIG. 2 in that the mounting armature 90 includes a first post 92 extending from the clamp 91. A first arm 93 is coupled to the first post 92 at one end of the first arm 93 and to an articulated scissor joint 98 at the opposite end of the first arm 93. A vertical post 99 is also coupled to the articulated scissor joint 98. Therefore, using the articulated scissor joint 98, the vertical post 99 can be positioned relative to the first arm 93 and the first post 92. The first arm 93 can be coupled to the first post 92 via a clamping joint 96. As described above, the upward tensioning rail 30 can be secured or removably coupled to the vertical post 99. As previously described, the mounting armature 90 can be used to position the upward tensioning rail 30 relative to the surgical field. It will be appreciated that the mounting armature 90 provides similar positioning as the mounting armature 20 with a simpler structure having decreased ranges of motion. Therefore, the mounting armature 90 in FIGS. 3-5 may allow for comparatively less precise positioning of the upward tensioning rail 30 than that shown in the embodiment of FIG. 2.

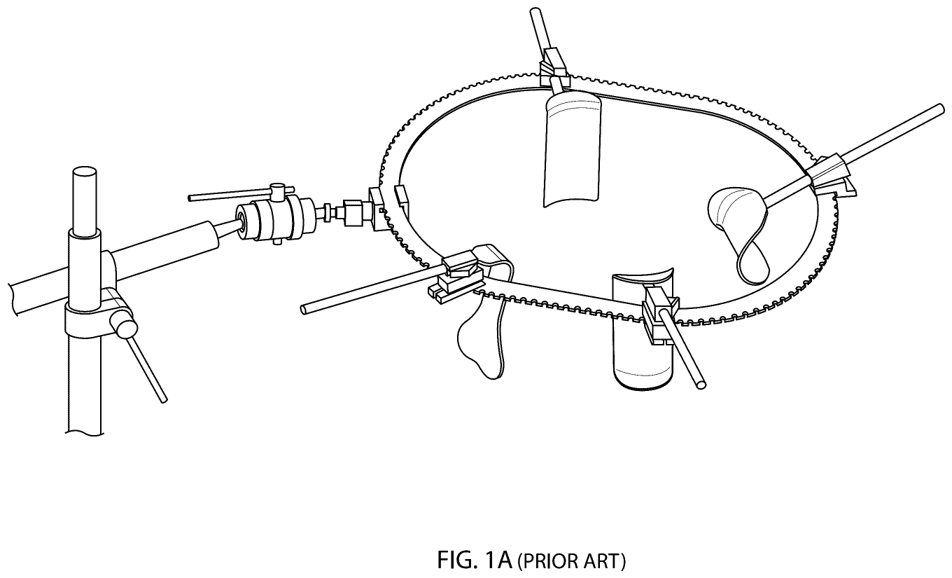

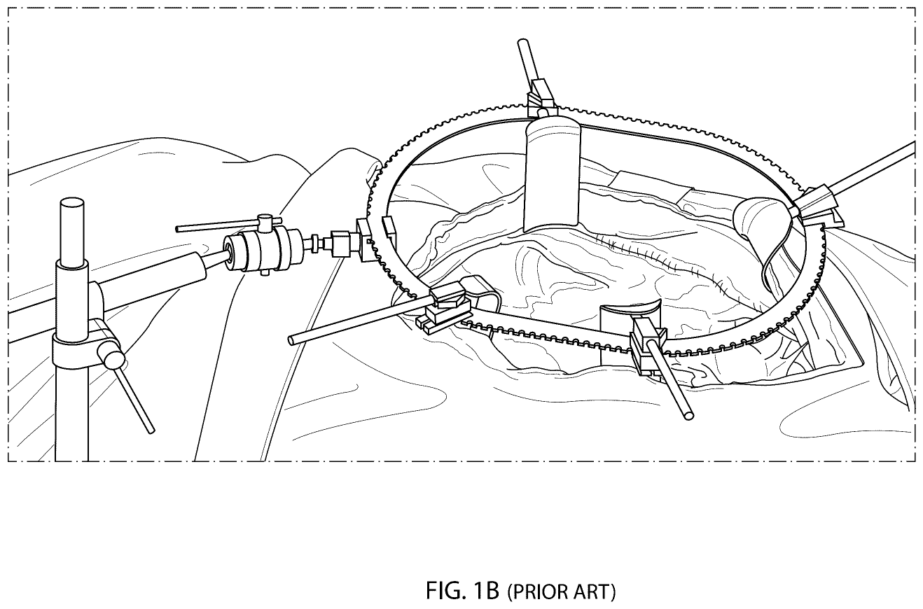

Referring to FIGS. 1-5, the upward tensioning rail 30 will be described. In one embodiment, the upward tensioning rail 30 may include a first rail member 32 and side rail members 34a and 34b that extend away from the first rail member 32 at an angle. It will be appreciated that when positioned, the side rail members 34a and 34b may extend toward (i.e., so as to surround) the incision site such that the upward tensioning rail 30 is substantially C-shaped. Alternatively, the upward tensioning rail 30 may have a curvature such that entire first member 32 is arcuate or C-shaped and does not have discrete side rail members. The upward tensioning rail 30 may have a wall 36 extending upward. The upward-extending wall 36 may extend upward from (e.g., from the edge of a generally horizontal wall of the first rail member 32 and the side rail members 34a and 34b). The upward-extending wall 36 may be substantially orthogonal to the horizontal wall of the first rail member 32 and side rail members 34a and 34b. As such, the profile of the upward tensioning rail 30 may be substantially L-shaped.

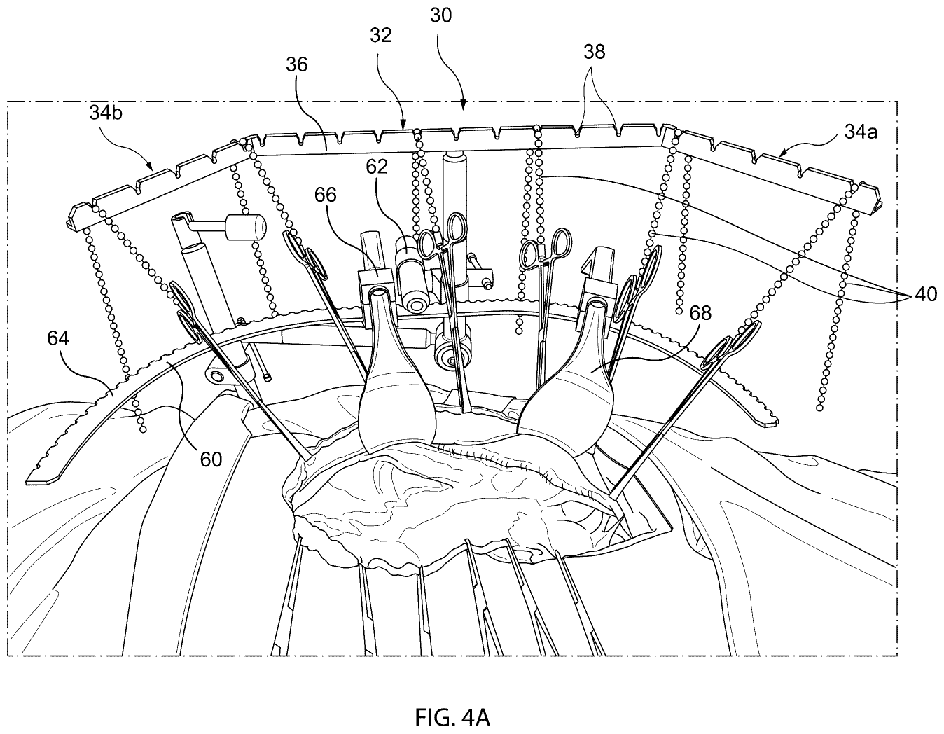

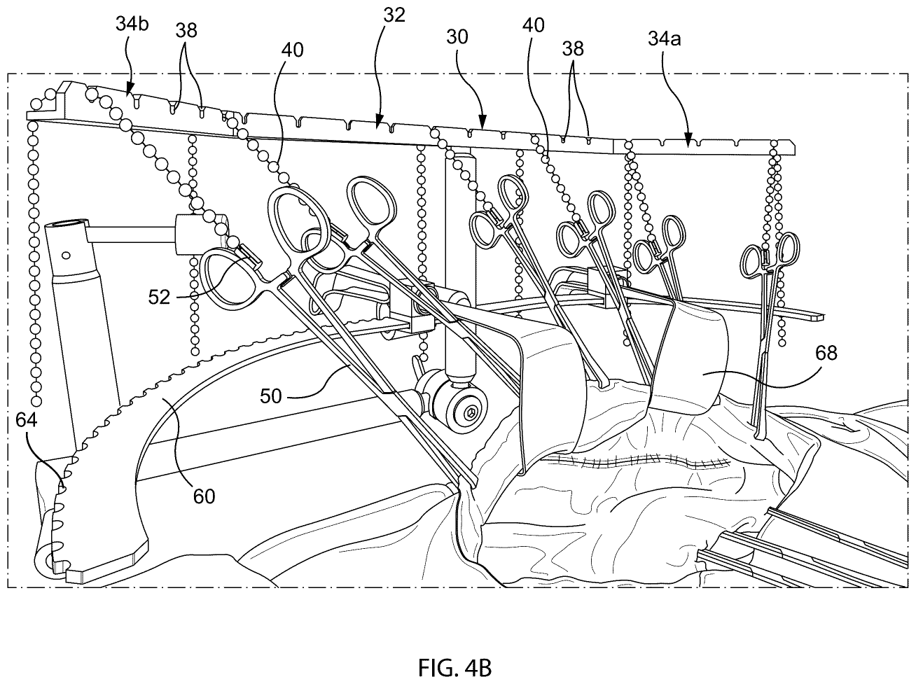

To retain the tension applied to the abdominal wall, the wall 36 includes a plurality of detent notches 38 distributed and spaced along its length. The detent notches 38 are configured to receive and retain chains or cables to provide upward tension in the surgical field as will be described in further detail below. As such, a plurality of ball chains 40 are provided having a proximal end 42 and a distal end 44 opposite the proximal end 42. Surgical clamps 50 are coupled to the chains 40 at their distal ends 44 (i.e., toward the patient). In the illustrated embodiment, the surgical clamps 50 used are conventional Lahey clamp forceps. However, other clamps may be used. For example, Kocher clamps (shown in FIG. 3a) may be welded or otherwise fixed or fastened to the distal end 44 of the respective ball chains 40. In FIG. 3, the distal ends of the ball chains 40 are coupled to the respective clamps 50 in any suitable or conventional manner. For example, the clamps 50 may be coupled to the ball chains 40 via clasp elements that can be affixed or welded as seen in FIG. 3b. Alternatively, as seen in FIGS. 4a-b, the ball chains 40 can be coupled to respective clamps 50 via slotted sleeves 52 that are affixed (e.g. welded) to the clamps 50. As seen in FIG. 4B, the respective sleeves 52 are open toward a distal end facing away from the upward tensioning rail 30 and are slotted along their length and substantially closed at the proximal end of the sleeve facing the upward tensioning rail 30. Therefore, the slotted sleeves 52 are able to receive and confine a terminal ball at the distal end 44 of the ball chain 40 and pull against that chain 40 in a direction away from the rail 30.

Returning to FIG. 3, in use, the upward tensioning rail 30 is pre-positioned generally above and at a suitable height and orientation relative to the surgical site, opposite to from where the surgeon will operate. The surgeon or a surgical assistant affixes a plurality of clamps 50 to the abdominal wall at the surgical incision site in a conventional manner, in order to facilitate retraction of the abdominal wall. A respective ball chain 40 is coupled to each clamp 50, either prior to or after affixing the clamps 50 to the abdominal-wall flap at the incision site. The surgeon or assistant can pull on the ball chains 40 to apply tension to the ball chains 40. When desired retraction is achieved, each tensioned ball chain 40 can be placed in a respective detent notch 38 in the upward tensioning rail 30. The engagement of the balls on the ball chain 40 with the notch 38 retains the position of the ball chain 40 relative to the upward engagement rail 30 and maintains consistent retraction tension. The magnitude of upward tension can be adjusted by adjusting the location along the ball chain's 40 length where it is inserted within the associated detent notch 38. Further, the angle vector of upward tension can be adjusted by positioning, or re-positioning, the upward tensioning rail 30 via adjustment of the mounting armature 20. As will be appreciated, tension may be incrementally increased while the surgery proceeds simply by adjusting the location along the chain 40 where it is received within the detent notch 38; i.e., shortening the segment thereof between the upward tensioning rail 30 and the associated clamp 50. By applying a plurality of clamps 50 to retract the abdominal wall at periodic locations along the length of the incision, where each claim 50 is drawn in upward tension via a respective ball chain 40 engaged with a detent notch 38 in the upward tensioning rail 30, persistent and reliable upward retraction of the abdominal wall is provided.

Notably, it is desirable to position the upward tensioning rail 30 generally opposite from where the operator will work during the procedure, relative to the surgical field, because in this manner the abdominal wall will be retracted upward and generally away from the operator, thus exposing the underlying abdominal fascia to him/her. As previously described, the upward tensioning rail 30 follows a generally C-shaped or arcuate path, such as or approximating a semi-circle, to provide available tensioning vectors from generally around (and not simply behind) the surgical field relative to the surgeon's vantage point. Alternatively, as shown in FIGS. 2 and 3, the upward tensioning rail 30 is composed of a number of linear rail members 32, 34a, 34b representing or approximating an open arc to provide the same benefit. But unlike conventional surgical-retractor systems, the upward tensioning rail 30 is not a closed shape, such as a circle or oval that completely surrounds the surgical field, to prevent interference with the surgeon's access to the upward-retracted abdominal wall for surgery.

As noted, upward retraction of the abdominal wall is important during AWR procedures, especially when operating on the abdominal fascia adjacent to the surgical incision. However, as the procedure continues and the surgeon must dissect deeper along the abdominal wall toward the spine, an additional lateral component of retraction can be desirable to provide deeper access. Further, additional lateral retraction also can be desirable when operating on larger or obese patients, to better visualize the operative target of the abdominal-wall fascia. Accordingly, as seen in FIGS. 2, 4a, and 4b, the retractor system 10 can include a lateral tensioning rail 60. The lateral tensioning rail 60 may also be affixed to the mounting armature 20. For instance, the lateral tensioning rail 60 may be configured to engage a clamp 62 on the vertical post 29, 99 as shown in FIGS. 3-5. Alternatively, the lateral tensioning rail 60 may be integrated into the vertical post 29, 99 as shown in FIG. 2. Similar to the upward tensioning rail 30, the lateral tensioning rail 60 can follow a generally arcuate path and be composed of a single curved rail or number of linear rail members that together form or approximate curve or a C-shape configured to partially surround the surgical site. Further, the lateral tensioning rail 60 can have a wall extending vertically from an edge of a horizontal wall of the rail 60, so that the lateral tensioning rail 60 has an L-shaped profile with detents configured to receive ball chains similar to that shown and described above with respect to the upward tensioning rail 30. In the illustrated embodiment, the lateral tensioning rail 60 is a curved rail with a flat profile.

The lateral tensioning rail 60 is configured to supply lateral retraction similar to a conventional Bookwalter retractor. As seen in the disclosed embodiment in FIGS. 2, 4a, and 4b the lateral tensioning rail 60 can be similar to that of a conventional Bookwalter retractor system except that the lateral tensioning rail 60 is not an enclosed shape. In this configuration, the lateral tensioning rail 60 can have detents 64 spaced along its length and distributed along the edge of the rail facing away from the surgical field. The detents 64 can be configured to engage selectively positionable clamps 66 that are configured to hold retractors 68, for instance Richardson retractors. The clamps 66 can be positioned on the lateral tensioning rail 60 and retained in place by engagement between the detents 64 and the clamps 66 in a conventional manner. The retractors 68 are slidingly engaged with the clamps 66 to provide lateral movement of the retractors toward or away from the incision site. The sliding movement of the retractors 68 allow the surgeon or assistant to apply lateral tension to the abdominal wall. Alternatively, the lateral tensioning rail 60 can be similarly configured as the upward tensioning rail 30 described above. As such, the lateral tensioning rail 60 can be configured to accommodate and provide selective tension to ball chains affixed to respective clamps to apply lateral forces to retract the abdominal wall. As described above, the lateral tensioning rail 60 is configured as an open arc, or it can be a series of linear segments approximating such an arc, mounted to the vertical post, and disposed generally opposite to the surgeon's position relative to the surgical field. Again, this ensures that the lateral tensioning rail 60 does not interfere with the surgeon's access to the abdominal fascia, which in AWR procedures is the surgical field and is not simply to be moved aside to access an underlying field.

As noted above, the upward tensioning rail 30 is disposed generally above the surgical field and the incision site in order to provide upward tension. Conversely, the lateral tensioning rail is located (e.g., fixed to the vertical post 29) at a lower position than the upward tensioning rail 30. For instance, the lateral tensioning rail 60 may be adjacent to and optionally substantially in a tangent plane relative to the incision site in order to supply lateral retraction. The clamp 62 can be selectively positioned on the vertical post 29, 89 to allow the lateral tensioning rail 60 to be positioned at an optimal height. Herein, “lateral” retraction does not necessarily mean retraction along a truly horizontal vector. Rather, it means that the vertical component of the retraction vector supplied from the lateral tensioning rail 60 will be smaller than that which is supplied via retraction from the upward tensioning rail 30. In some embodiments, the retraction vector from the lateral tensioning rail 60 will be along an angle that is up to 10° or 20° from the horizontal. As with upward tension described above, the lateral-tension vector also can be set or adjusted by positioning the lateral tensioning rail 60 appropriately to correspond to the desired vector. It will be appreciated that in systems utilizing both an upward tensioning rail 30 and a lateral tensioning rail 60, the upward and lateral tensioning rails 30, 60 may extend in respective, substantially parallel planes.

FIGS. 4a-4b show the abdominal-wall retraction system including both an upward tensioning rail 30 and a lateral tensioning rail 60, supplying both upward tension and lateral tension to the abdominal wall from the incision site during an AWR procedure. This ensures both exposure of the underlying abdominal fascia for surgical access, as well as sufficient lateral retraction to expose deeper portions of that fascia, and/or to retract excess tissue as may be present in larger or obese patients. The combination of both upward and lateral tension as described here has been found to provide desirable, sustained surgical access to the abdominal fascia for hernia repairs in obese patients, while eliminating problems associated with surgical-assistant fatigue.

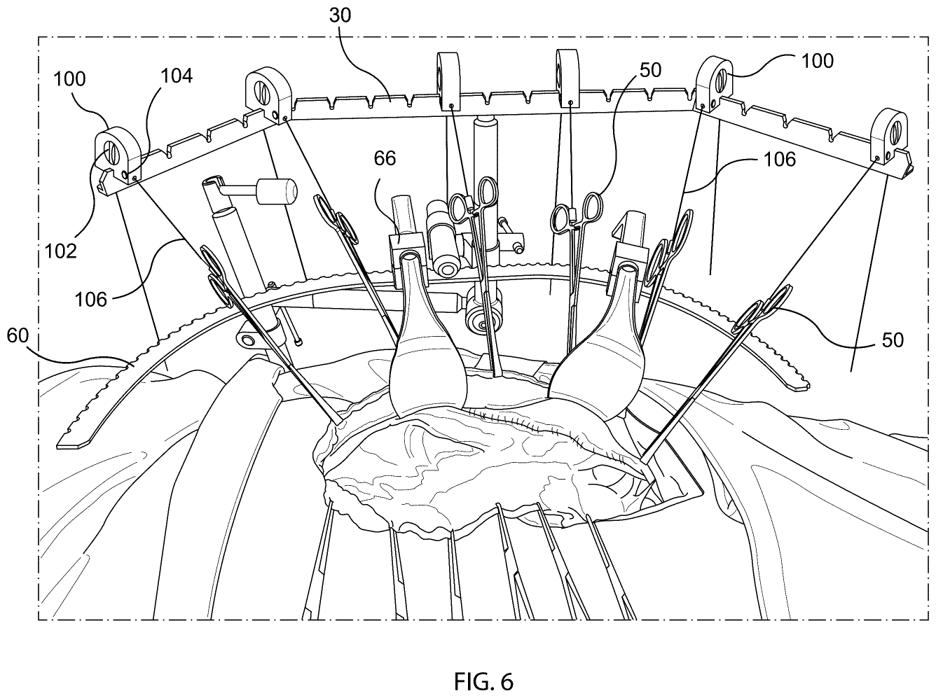

In embodiments of the abdominal retraction system 10 described above, upward tension is supplied via ball chains affixed to conventional clamps as described. However, the upward tensioning rail 30 may be provided using other tools similar to that of a conventional Bookwalter retractor system, e.g., similar retractors, clamps, etc. In an alternative embodiment shown in FIG. 6, one or both of the tensioning rails may instead be fitted with fixed or adjustable-position indexing rollers 100 (e.g., ratcheted rollers), configured to wind respective cords, cables, or wires 106 thereon, which can be fitted or affixed to suitable clamps at their distal ends. Then tension could be adjusted by winding the respective cords, cables, or wires 106 and fixed by a conventional ratcheting mechanism associated with each roller. As an example, each roller could have a handle or knob 102 configured to rotate to apply tension to a respective wire 106 and a release button 104 configured to release the applied tension to the wire, e.g., using a conventional ratcheting mechanism. Alternatively, the roller 100 can be configured to allow an operator to apply tension to the wire 106 by pulling the proximal end of the wire 106. Once the surgeon releases the tension on the wire, either a ratcheting mechanism or a friction cam can engage the wire to maintain the tension applied by the surgeon or assistant.

As will be appreciated, the contemplated retractor system 10 is not limited to ball chains, retractor arms (as in a conventional Bookwalter system), or wires to supply tension to clamps from the respective tensioning rails 30, 60. These and other known or conventional tension linkages configured to supply tension between the respective rail and an associate clamp can be used. Nor is the retractor system 10 limited to clamps for affixation to the abdominal wall to supply tension. Other conventional surgical tools like retractors can be tensioned from either or both of the tensioning rails 30, 60 to supply the retraction herein described. The described clamps are merely examples of such surgical tools that can be used to engage the incision site.

The retractor system 10 as described so far above relates to retracting the abdominal wall in order to provide access to operate on (e.g., dissect) the abdominal fascia. However, as that fascia is dissected from the rectus and transversus abdominus muscles of the abdomen, it may be desirable to supply counter tension. For instance, the surgeon or assistant may need to apply tension in a direction that tends to stretch the dissected posterior rectus fascia taut and away from the retracted abdominal wall. Maintaining tautness (i.e., planar tension) in the dissected fascia can be important as the surgeon continues to operate and further dissect it from the rectus and transversus abdominus muscles in hernia surgery.

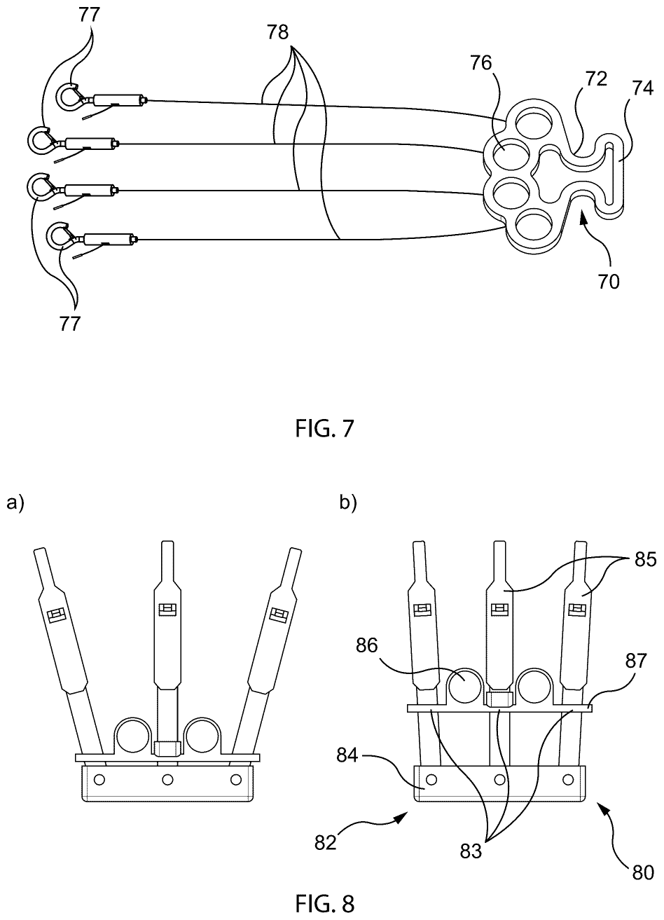

FIGS. 7-9 illustrate embodiments of counter-tension clamping assemblies configured to grasp the dissected abdominal fascia and to allow the surgeon to draw the fascia away from the rectus and transversus abdominus muscles and maintain tautness therein. The direction of such counter-tension will generally be toward the surgeon and away from the tensioning rails 30, 60 noted above. Notably, this tension must be supplied over a broad area, in a consistent fashion, with both medial and downward retraction. Currently this is performed with the surgeon's non-operative hand. The force and consistency of such counter-tension may be severely limited based on the dexterity of the surgeon, and the size of the hand. Surgeons with smaller hands may be unable to expose the entirety of the abdominal wall and are forced to operate on small areas, increasing operating room time and potentially damage to the dissecting structures due to inadequate exposure.

In FIG. 7, the counter tension clamping assembly 70 includes a handle 72 having a palm bar 74 configured to rest against the palm of a hand. The clamping assembly 70 further includes finger holes 76 opposite the palm bar 74, in a location that facilitates placing one's fingers through the holes 76 to make a fist and pull the palm bar 74 against the palm. A plurality of tension 78 wires extends from the handle 72 near the finger holes 76. At the proximal end of the tension wires 78, the tension wires 78 are secured to the handle 72, for instance, by welding. Alternatively, the wires 78 may be clamped, fastened, or otherwise suitably secured to the handle 72. A clamp 79 is affixed to the distal end of each tension wire 78. The clamps 79 are configured to be affixed to the dissected abdominal fascia. Alternatively, the distal end of each wire may include a mounting means allowing the operator to customize the counter tensioning assembly 70 by being configured to selectively remove the tool at the distal end of the wire. For instance, as illustrated in FIG. 7, a hook 77 having a snap closure can be affixed to the distal end of each tension wire to allow for easy removal and installation of a certain style of clamp or other tool.

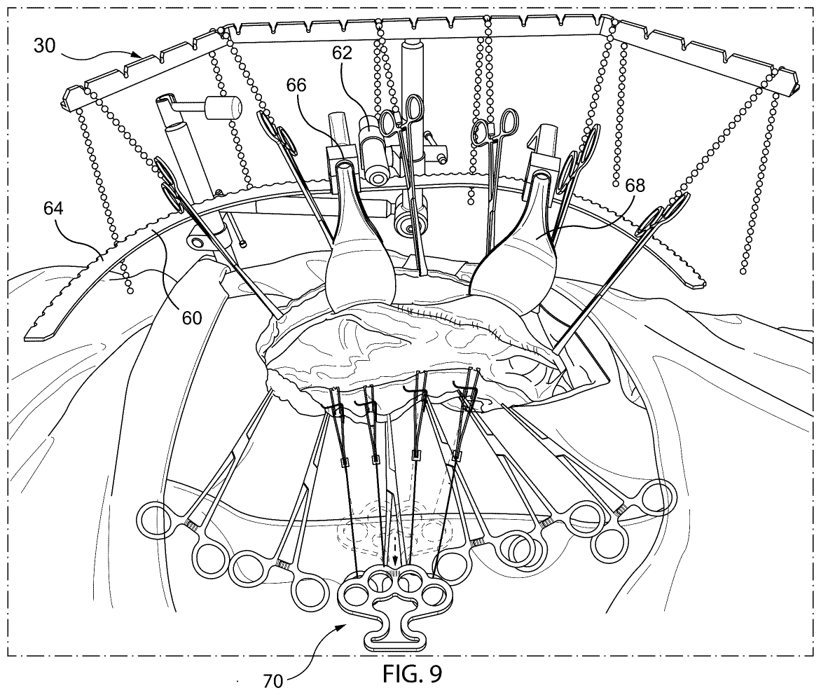

Turning to FIG. 9, in use, the surgeon (or a surgical assistant) can affix the clamps 79 to (or adjacent to) the terminal edge of the dissected portion of the abdominal fascia and then pull in a direction substantially opposite to, or counter to, the tension vectors supplied to the abdominal wall from the upward tensioning rail 30 and/or the lateral tensioning rail 60 in order to separate the fascia from the rectus and transversus abdominus muscles and maintain its tautness while the surgeon operates. Notably, this counter-tension step is a manual one. The clamping assembly 70 enables a single hand to manipulate multiple (four are shown) separate clamps that can be affixed to the fascia at spaced locations, in order to provide uniform tension with a single, non-operating hand. Moreover, the fascia is essentially weightless, and counter-tension will not be supplied against a significant weight or opposing elastic force as in the case of retracting the abdominal wall. So, the degree of user fatigue is substantially lower than in the case of manual abdominal-wall retraction.

Alternatively, FIG. 8a-8b illustrates another embodiment of a counter-tension clamping assembly 80, which similarly includes a handle 82 with a palm bar 84 and finger holes 86. A plurality of clamping forceps 85 are pivotally cantilevered from the palm bar 84. The clamping forceps 85 may be biased in an open position and can manually be closed to clamp to the terminal edge of the dissected portion of the abdominal fascia. While the clamping assembly 80 is illustrated with three clamping forceps 85, it will be appreciated that the clamping assembly 80 can include and number of two or more clamping forceps 85. In this embodiment, the finger holes 86 are provided in a separate actuation bar 87 that is movable relative to the palm bar 84. The actuation bar 87 may include a number of fixed openings 83 that corresponds to the number of clamping forceps 85. The openings 83 are configured to receive a portion of the clamping forceps 85 such that the actuation bar 87 can slide toward and away from the palm bar 84 over or relative to the clamping forceps 85. The handle 82 and the palm bar 84 may be contoured arches to improve the ergonomics of the counter-tension clamping assembly 80.

The movement of the actuation bar 87 moves the clamping forceps 85 by either splaying them apart or drawing them together. Since the clamping forceps 85 are pivotally attached to the palm bar 84, the movement of the actuation bar 87 effectively changes the distances between the clamping ends of the clamping forceps 85. As will be appreciated, as the user draws the actuation bar 87 toward the palm bar 84 by closing his/her fist, the clamping forceps 85 will be caused to splay by the openings 83 in the actuation bar 87 so that the distance between the clamping ends in increased as shown in FIG. 8a. Whereas, when the user opens his/her fist thereby separating the actuation bar 87 from the palm bar 84, the clamping forceps 85 will be drawn together by the openings 83 in the actuation bar 87 so that the distance between the clamping ends decreases and the clamping ends are similarly drawn together as shown in FIG. 8b The counter-tension clamping assembly 80 of FIG. 8 can be used in a similar way as described for that shown in FIG. 7, except that here the user is empowered to not only supply pulling tension as already described, but also to adjust a lateral tension in the clamped fascia by adjusting the degree to which the clamping forceps 85 are splayed. Therefore, the counter-tension assembly 80 can provide counter-tension to the fascia and also flatten the fascia. The ability to adjust pulling and lateral tension in the fascia can improve fine-tuning the counter-tensioning adjustment desired to facilitate an efficient operation.

The system has been described with reference to example embodiments. Modifications and alterations thereto will be evident to persons of skill in the art upon a reading and understanding this specification.

What is claimed is:

1. An abdominal-wall retractor comprising:
a mounting armature;
an upward tensioning rail affixed to the mounting armature; and
one or more upward tension linkages configured to provide tension between the upward tensioning rail and one or more respective upward retraction tools;
wherein the mounting armature is configured to hold the upward tensioning rail in a fixed position above a surgical site in order to retract an abdominal wall upward, when grasped by the upward retraction tool(s), at an angle of at least 20° from horizontal via the provided tension in the one or more upward tension linkages during a surgical procedure.

2. The retractor of claim 1, the upward tensioning rail having or approximating an open arcuate shape.

3. The retractor of claim 1, the upward tension linkage comprising a ball chain and the upward retraction tool comprising a clamp.

4. The retractor of claim 1, the one or more upward tension linkages comprising a cord or wire.

5. The retractor of claim 1, wherein the tension supplied by the upward tension linkage is adjustable.

6. The retractor of claim 1, comprising a plurality of said upward tension linkages configured to provide upward tension for retraction at different locations, respectively, along an abdominal wall flap during a surgical procedure.

7. The retractor of claim 1, the upward tensioning rail comprising detents distributed along a length thereof, the detents being configured to adjustably engage and retain the upward tension linkage(s).

8. The retractor of claim 7, the upward tensioning rail comprising a vertical wall extending upward from a generally horizontal wall thereof, the detents being formed in the vertical wall.

9. The retractor of claim 4, further comprising one or more ratchet wheels removably positioned on the upward tensioning rail and operatively engaged with the respective one or more cords or wires, the one or more ratchet wheels being configured to increase the tension between the upward tensioning rail and the upward retraction tools as the ratchet wheels are rotated.

10. The retractor of claim 1, further comprising:
a lateral tensioning rail affixed to the mounting armature at a location lower than the upward tensioning rail; and
a one or more lateral tension linkages configured to provide tension between the lateral tensioning rail and one or more second lateral retraction tools;
wherein the position of the lateral tensioning rail on the mounting armature results in lateral retraction of the abdominal wall via the provided tension in the one or more lateral tension linkages during the surgical procedure.

11. The retractor of claim 10, the lateral tensioning rail having or approximating an open arcuate shape.

12. The retractor of claim 10, comprising a plurality of said lateral tension linkages configured to provide lateral tension for retraction at different locations, respectively, along an abdominal wall flap during a surgical procedure.

13. The retractor of claim 10, said upward tensioning rail and said lateral tensioning rail extending in respective, substantially parallel planes.

14. The retractor of claim 1, wherein the mounting armature is configured to be affixed to an operating table or to a stationary stand near the operating table.

15. An abdominal-wall retractor system comprising:
a mounting armature;
an upward tensioning rail affixed to the mounting armature;
an upward tension linkage configured to provide upward tension between the upward tensioning rail and an upward retraction tool along an upward retraction vector;
a lateral tensioning rail affixed to the mounting armature at a location lower than the upward tensioning rail;
a lateral tension linkage configured to provide lateral tension between the lateral tensioning rail and a lateral retraction tool along a lateral retraction vector;
wherein the upward retraction vector is at a greater angle than the lateral retraction vector relative to horizontal.

16. The system of claim 15, comprising means for adjusting the upward tension, and means for adjusting the lateral tension.

17. The system of claim 15, wherein the upward retraction vector is at an angle of at least 20° from the horizontal.

18. A method for abdominal-wall repair, comprising:
applying an upward tension to an abdominal wall flap via an upward tension linkage extending between an upward tension rail and the abdominal wall flap along an upward retraction vector at an angle of at least 20° relative to horizontal, in order to expose underlying fascia of the abdominal wall flap.

19. The method of claim 18, further comprising:
applying a lateral tension to the abdominal wall flap along a lateral retraction vector at an angle relative to horizontal less than 20°.

20. The method of claim 19, said upward retraction vector being at an angle of at least 30° relative to horizontal.
